(19) United States
(12) Patent Application Publication
(10) Pub. No.: US 2004/0133128 A1
(43) Pub. Date: Jul. 8, 2004

(54) Assay device with attachable seal for use with specimen collection and assay containers

(76) Inventors: Zhumin Guan, Hangzhou (CN); Yuchang Wu, Hangzhou (CN); Ying Yang, Hangzhou (CN)

Correspondence Address: FISH & RICHARDSON, PC, 12390 EL CAMINO REAL, SAN DIEGO, CA, 92130-2081, US

(21) Appl. No.: 10/336,203

(22) Filed: Jan. 4, 2003

(51) Int. Cl. A61B 5/00

(52) U.S. Cl. 600/584

(57) ABSTRACT

The present invention includes but is not limited to an analysis device having a housing, an assay strip and a seal attachable to the housing. The present invention is inserted into a specimen collection and assay container such as a urine collection and analysis cup. The housed assay strip is sealed within the collection container. If the container has a separate reservoir for testing a portion of the sample, the housed assay strip may be sealed inside the reservoir. The housing of the assay device is attachable to the seal by a variety of means, for example, by prongs, clips or glue.

### TECHNICAL FIELD

The present invention relates generally to the field of sample collection and assaying devices, which can be used to manipulate samples, including samples used to assay for analytes, especially drugs of abuse, antibodies, antigens and biological moieties such as glucose. More specifically the present invention relates to a test device with an attachable seal for use in a sample collection and assay container.

### BACKGROUND

A variety of sample collection and assay devices for clinical or home use are available and described in the literature. These devices can be used for an assortment of purposes, including the detection of drugs or biological compounds such as glucose or hormones, antibodies or etiological agents. Many of these devices are complex in design and are difficult to use. Also, these devices are often manufactured and fabricated from relatively expensive materials. The present invention addresses these problems, and provides related benefits.

### SUMMARY

The present invention includes, but it not limited to, an analysis device able to reversibly engage a sample collection device, such as, for example, a urine collection and assay cup. The present invention may engage a primary chamber such as one that accepts a sample or may engage a reservoir, a secondary chamber that receives a sample from the primary chamber. The analysis device of the present invention includes a housing, an assay strip positioned within the interior of the housing, a seal able to seal the housing within a collection container; and an attachment means able to attach the seal to the housing. The assay strip may detect the presence or measure the concentration of at least one analyte of interest, such as, but not limited to a drug of abuse, a hormone, a protein, a nucleic acid molecule, a metabolic product, a pathogen, and the like. The present invention includes a variety of other useful aspects, which are detailed herein.

### BRIEF DESCRIPTION OF THE FIGURES

FIG. 1 depicts one aspect of the present invention including assay strips encased in a housing 101 and attachable to a seal 200. In this instance prongs are used as an attachment means 104 to attach the housing 101 to the seal 200. The dashed lines illustrate one example of a specimen collection and assay container 300 including a reservoir 302 able to accept the present invention 100. The housing 101 is of a size and shape that allows it to be inserted into the reservoir 302. The seal 200 is of a size able to seal the housing air tight within the reservoir 302 when engaged.

FIG. 2 depicts the back of one aspect of a housing 101 of the analysis device of the present invention 100. The depicted housing 101 includes an attachment means 104 positioned at about the top 102 of housing 101. Pins 106 and notches 105 substantially in or near the bottom 103 of housing 101 are also shown.

FIG. 3 illustrates the present invention 100 being inserted into a reservoir 302. The reservoir seal 200 is depicted as attached to the top of the housing 101. The ribs 201 on the seal 200 conform to the top interior of the reservoir 302, allowing the housing 101 to be seal air tight within the reservoir 302 once engaged, thereby controlling an influx of a sample into the reservoir 302.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down” or “upper” or “lower” and the like refer to orientation of the parts during use of the device. Where a term is provided in the singular, the inventors also contemplate the plural of that term. The nomenclature used herein and the laboratory procedures described below are those well known and commonly employed in the art. As employed throughout the disclosure, the following terms, unless other wise indicated, shall be understood to have the following meanings:

“Assaying” denotes the qualitative or quantitative testing a sample for an analyte. Assaying may incorporate an immunological test, a chemical test, an enzymatic test and the like. The present invention may assay for the presence or concentration of a variety of analytes such as but not limited to, a chemical, an organic compound, an inorganic compound, a metabolic product, a drug or a drug metabolite, an organism or a metabolite of such an organism, a nucleic acid, a protein, a hormone or a combination thereof. Assaying may involve comparing the results obtained against a positive or negative control as is common in the biochemical and immunological arts. When determining the concentration of an analyte the assay may also include at least one quantitative control to determine the amount of analyte present and may further include mathematical calculations such as comparing the amount of analyte to the volume within the collection container or reservoir.

An element of the present invention is “integral to” another element of the present invention when the two elements are manufactured or provided as a single piece.

An element of the present invention is “separate from” another element of the present invention when the two elements are manufactured or provided as separate pieces.

A “reagent” can be any chemical, including organic compounds and inorganic compounds and combinations thereof. A reagent can be provided in gaseous, solid, or liquid form, or any combination thereof, and can be a component of a solution or suspension. A reagent preferably includes fluids, such as buffers useful in methods of detecting analytes in a sample or specimen, such as anticoagulants, diluents, buffers, assay reagents, specific binding members, detectable labels, enzymes and the like. A reagent can also include an extractant, such as a buffer or chemical, to extract an analyte from a sample or specimen or a sample collection device. For example, a buffer can be used to extract analytes from the sample or specimen, such as LPS from bacteria.

An “assaying device” or “assay device” is a device for detecting the presence and/or concentration of an analyte in a sample or specimen. Assay devices of the present invention include but are not limited to lateral flow detection devices such as assay strip devices, and columns. The assay device may refer to the above referenced lateral flow detection device or a lateral flow detection device encased in a housing. In lateral flow detection devices the liquid sample or specimen moves through a matrix or material by lateral flow or capillary action, such as an immunochromatographic device. This migration generally involves passage through a sample application zone, a reagent zone and a detection zone. The sample application zone being the region of the lateral flow detection device which is contacted first by the sample, the reagent zone being the region in which particular reagents for the desired assay are positioned such that they may migrate with an analyte along the device, such as for example a mobilizable labeled antibody, and the detection zone being the region in which the results of the assay are visualized or determined, such as for example a region containing an immobilized antibody to capture the analyte. A lateral flow detection device may be used in a substantially vertical or a substantially horizontal orientation or in an orientation substantially between vertical and horizontal. Preferably, neither a reagent zone nor a detection zone should contact the specimen or analyte unless the specimen or analyte migrates along the lateral flow detection device. However the present invention recognizes and encompasses embodiments where reagents such as but not limited to mobilizable antibodies are positioned such that they contact the sample or specimen without migration along the assay strip. That is, reagents may be contained within a sample application zone. This may eliminate the need for an independent reagent zone. Persons knowledgeable in the art commonly refer to a lateral flow detection device using terms such as “immunochromatographic,” “dip sticks,” “membrane technology” and “test strips.”

“Analyte” is the compound or composition to be detected or measured. An analyte is generally capable of binding to a ligand, a receptor, or an enzyme. The analyte may be an antibody or antigen such as a protein or drug, or a metabolite. The precise nature of antigenic and drug analytes together with numerous examples thereof are disclosed in U.S. Pat. No. 4,299,916 to Litman, et al., particularly columns 16 to 23, and in U.S. Pat. No. 4,275,149, particularly columns 17 and 18, the disclosures of which are incorporated herein by reference in their entirety. Analytes can include antibodies and receptors, including active fragments or fragments thereof. An analyte can include and analyte analogue, which is a derivative of an analyte, such as, for example, an analyte altered by chemical or biological methods, such as by the action of reactive chemicals, such as adulterants or enzymatic activity. An analyte can be but is not limited to a drug, a drug of abuse, a hormone, a protein, a nucleic acid molecule, an etiological agent, a specific binding member.

An “antibody” is an immunoglobulin, or derivative or active fragment thereof, having an area on the surface or in a cavity, which specifically binds to and is thereby defined as complementary with a particular spatial and polar organization of another molecule. Antibodies encompassed by the present invention include but are not limited to IgG, IgM, IgE, Fe, F(ab), F(ab)′2, light chain such as kappa and lambda, heavy chain fragments and the like. The antibody can be monoclonal or polyclonal and can be prepared by techniques that are well known in the art such as, for example, immunization of a host and collection of sera or hybrid cell line technology. Determining the proper antibody may be performed by performing binding assays known in the immunological arts such as an ELISA with the analyte of interest.

A “sample” or “specimen” refers to any material to be assayed for the presence and/or concentration of an analyte. Preferably, a sample is a fluid sample such as a liquid sample. Examples of liquid samples that may be assayed using include bodily fluids such as but not limited to blood, serum, plasma, saliva, urine, ocular fluid, semen, and spinal fluid; water samples, such as samples of water from oceans, seas, lakes, rivers, and the like, or samples from home, municipal, or industrial water sources, runoff water or sewage samples; and food samples, such as milk or wine. Viscous liquid, semi-solid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof, as, for example a suspension of lysed or unlysed cells in a buffer or solution. Samples can comprise biological materials, such as cells, microbes, organelles, and biochemical complexes. Liquid samples can be made from solid, semisolid or highly viscous materials, such as soils, fecal matter, tissues, organs, biological fluids or other samples that are not fluid in nature. For example, these solid or semi-solid samples can be mixed with an appropriate solution, such as a buffer, such as a diluent or extraction buffer. The sample can be macerated, frozen and thawed, or otherwise extracted to form a fluid sample. Residual particulates may be removed or reduced using conventional methods, such as filtration or centrifugation.

Other technical terms used herein have their ordinary meaning in the art that they are used, as exemplified by a variety of technical dictionaries.

The Introduction

As a non-limiting introduction to the breath of the present invention, the present invention includes several general and useful aspects, including but not limited to an analysis device able to reversibly engage a sample collection device optionally having a reservoir, the analysis device including a housing, an assay strip, a seal, and an attachment means. The seal may be attached to the top of the housing by the attachment means such that the assay device can be sealed inside the specimen collection device and optionally within the reservoir. The assay strip assays a portion of the specimen or sample for an analyte.

These aspects of the invention, as well as others described herein, can be achieved by using the articles of manufacture and compositions of matter described herein. To gain a full appreciation of the scope of the present invention, it will be further recognized that various aspects of the present invention can be combined to make desirable embodiments of the invention. In addition, a variety of other aspects and embodiments of the present invention are described herein.

Housing and Assay Strips

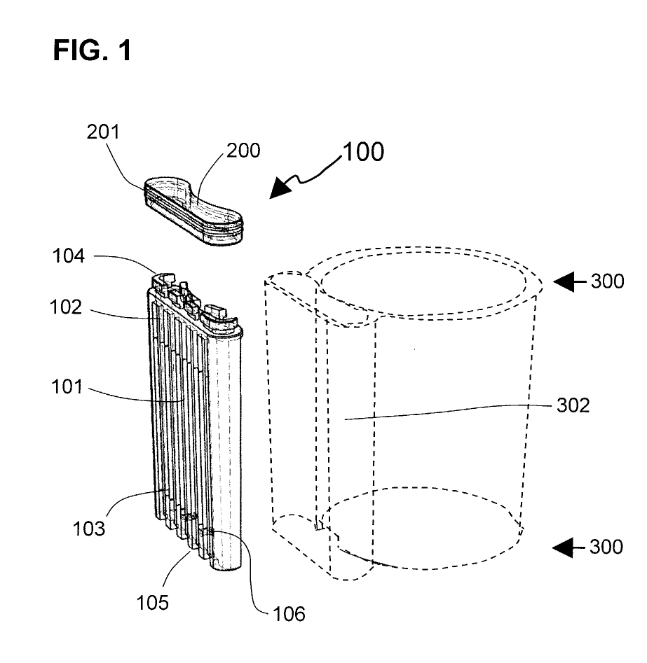

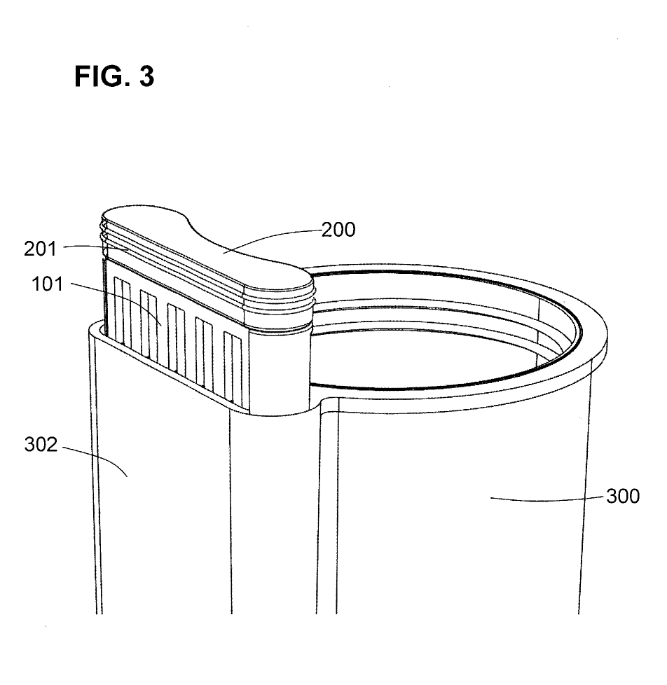

With reference to FIG. 1 through FIG. 3, the present invention 100 includes a housing 101 and assay device such as an assay strip able to be sealed within a sample collection container 300 and able to detect the presence or concentration of an analyte. When the sample collection container includes a reservoir 302, the assay device may be sealed within the reservoir 302. The housing 101 has a top end 102 and a bottom end 103, and an exterior and an interior. The assay strip being positioned within the housing. The housing 101 of the present invention may provide support for the assay strip, may provide a structure for which to suspend or reduce the movement of the assay strip within the sample collection device, and may provide a surface which indicia may be added such as the analyte to be assayed, control information such as positive and negative controls, or patient information such as name and date of test.

The housing 101 may be constructed from a rigid material or a pliable material. These materials can include but are not limited to metal, silicon, glass, ceramic, plastic and synthetic and natural polymers or any combination thereof. In one embodiment of the invention, the housing 101 can be manufactured from a polypropylene composite using an appropriate manufacturing method such as pressure injection molding or machining. In another embodiment the housing 101 is constructed from polystyrene. Methods of manufacturing the housing can include but are not limited to milling, casting, blowing, and spinning. One skilled in the art of manufacturing would recognize that construction of the housing 101 may involve molding then joining two halves such as by gluing, thermowelding and the like.

During production, an attachment means 104 and at least one pin 106 may be molded to the housing 101. As will further be discussed the attachment means 104 allows attachment of the housing 101 to the seal 200 and may be positioned at or near the top 102 of the housing and the at least one pin 106 may be used to assist in maintaining the housing 101 in a vertical orientation and may be positioned at or near the bottom of the device. However the attachment means 104 and at least one pin 106 may be formed then joined to the housing 101 when desired such as by gluing or thermowelding. The at least one pin 106 may be made of any material known in the art, such as, for example, metal, silicon, glass, ceramic, plastic or a polymer.

The housing 101 may further comprise at least one viewing window, such as those commonly employed in the art, for viewing the results of the assay. The viewing window may be an aperture or a transparent material such as a plastic or glass.

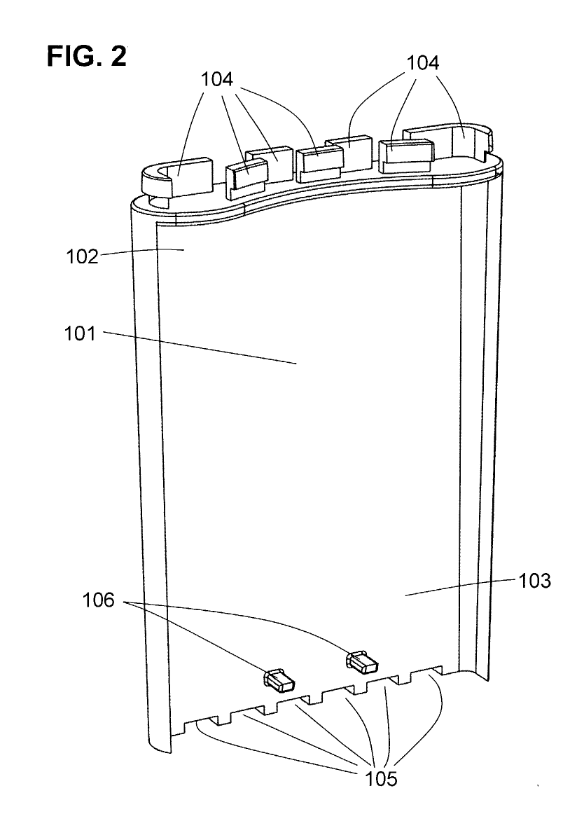

Referring to FIG. 2, substantially at or near the bottom 103 of the housing 101 at least one notch 105 may communicatively connect the exterior and the interior of the housing 101. The at least one notch 105 allows the sample to enter the housing 101 and contact an assay strip, preferably a sample application zone. In additional aspects of the present invention, the bottom of the housing 101 further includes a wick, preferably made of a bibulous material, which optionally assists the sample or specimen to enter the housing 101 and to travel to the assay strips located within the housing 101. When a wick is used it may further be used as a sample application zone directing the sample towards a reagent zone on the test device.

The assay device 100 of the present invention preferably comprises at least one lateral flow detection device such as an assay strip or test strip which can be any assay strip or test strip known in the art. Such lateral flow detection devices include, but are not limited to: immunoassays, chemical assays and enzymatic assays commonly known in the art, such as but not limited to, single antibody immunoassays, multiple antibody immunoassays, sandwich immunoassays, competitive immunoassays, non-competitive immunoassays and the like, including assays that utilize horseradish peroxidase, alkaline phosphatase, luciferase, antibody conjugates, antibody fragments, fluorescently tagged antibodies, modified antibodies, labeled antibodies, antibodies labeled with colloidal gold, antibodies labeled with colored latex bead, and the like, which are commonly known in the art. Examples of some assay strips that can be incorporated into the present invention can be found in the following U.S. patents: U.S. Pat. No. 4,857,453; U.S. Pat. No. 5,073,484; U.S. Pat. No. 5,119,831; U.S. Pat. No. 5,185,127; U.S. Pat. No. 5,275,785; U.S. Pat. No. 5,416,000; U.S. Pat. No. 5,504,013; U.S. Pat. No. 5,602,040; U.S. Pat. No. 5,622,871; U.S. Pat. No. 5,654,162; U.S. Pat. No. 5,656,503; U.S. Pat. No. 5,686,315; U.S. Pat. No. 5,766,961; U.S. Pat. No. 5,770,460; U.S. Pat. No. 5,916,815; U.S. Pat. No. 5,976,895; U.S. Pat. No. 6,248,598; U.S. Pat. No. 6,140,136; U.S. Pat. No. 6,187,269; U.S. Pat. No. 6,187,598; U.S. Pat. No. 6,228,660; U.S. Pat. No. 6,235,241; U.S. Pat. No. 6,306,642; U.S. Pat. No. 6,352,862; U.S. Pat. No. 6,372,515; U.S. Pat. No. 6,379,620; and U.S. Pat. No. 6,403,383 which are herein incorporated by reference. Further examples of some assay strips that can be incorporated into the present invention can be found in the following U.S. patent application Ser. Nos. 09/579,672; 09/579,673; 09/653,032; 60/233,739; 09/915,494, 10/211,199 and 09/860,408 and are herein incorporated by reference.

The one or more assay strips can be of any shape and dimensions, but preferably is a rectangular assay strip. The one or more assay strips can be used separately or can be arrayed on or in a common support such as an assay card then positioned within the housing 101. Preferably, multiple assay strips of an assay device can be arranged such that the sample application zones or regions of the assay strips can be contacted with the specimen in the reservoir 302 of the specimen collection container 300.

Assembly of the housing and assay strip may performed using a variety of methods such as manufacturing the housing in two halves and encasing the assay strip between the two halves. This technique may include positioning the assay strip along one half then affixing the second half to the first half thereby encasing the assay strip. Affixing the halves may be by any method known to those skilled in the art of medical device construction such as snapping, gluing, welding, thermo welding and the like. Each of the two halves may also have complimentary surfaces such as a pin and aperture to facilitate proper alignment of the halves. Furthermore the back of the assay strips may be glued to the housing to further prevent movement of the assay strips within the housing 101 and the assay strips may be aligned such that they are observable through a window or aperture in the housing 101. In another embodiment the assay strips are affixed to a rigid or pliable card then the card affixed to the first half of the housing 101. In another embodiment the assay strips are pinched when the two halves are affixed together. When the assay strips are pinched, they should be done so in a region that does not adversely affect the assay such as above the detection zone. Alternatively a card may be used as the housing 101.

Seal

In further embodiments of the present invention, an opening of a reservoir 302 is hermetically sealed or sealed air tight with a seal 200. Suitable seals can include, but are not limited to, plugs, films and self-adhesive seals made of paper, wax paper, plastic materials, thin metal films, or metallicized plastic or paper. In preferred embodiments, the seal 200 is a plug made of rubber-like plastic with deformable ribbing 201, which hermetically seals or seals air tight the reservoir 302. Preferably, the seal 200 is attached to the top 102 of the housing 101 by one of several attachment means 104 such as but not limited to gluing, taping, micro-welding, welding, pinning, stapling, snapping, strapping, clipping and the like. In preferred embodiments of the present invention, the seal 200 is attached to the top 102 of the housing 101 by at least one prong on the top 102 of the housing 101. In this embodiment a rim or a skirt is molded into the seal 200 and is positioned about the bottom of the seal 200 projecting generally inward. Attaching the housing 101 to the seal 200 may be accomplished by pressing the housing 101 and seal 200 together until the at least one prong snaps together with the rim or skirt. When the seal 200 is formed from a rubber like plastic material, the seal 200 can be removed from the housing 101 by peeling the seal 200 away from the housing 101 thereby releasing the at least one prong from contact with the rim or skirt. In alternative embodiments such as those which incorporate a housing having a narrow depth such as a test card, the seal 200 may attach to the housing 101 by a clipping structure allowing the housing 101 to be reversibly or irreversibly clipped to the seal 200.

Once the seal 200 is attached to the housing 101, the seal 200 engages the reservoir 302 or the sample collection container 300 thereby sealing the housing 101 within the reservoir 302 or specimen collection container 300. The resulting sealed reservoir 302 will permit only a fixed amount of a sample or specimen to enter the reservoir 302 due to the air pressure within the reservoir 302. However the present invention also envisions embodiments where the housing 101 is inserted in the reservoir 302 or sample collection container prior to attaching the seal 200 to the housing 101. In each of these embodiments, preferably the seal 200 and the opening or aperture to be sealed have complimentary shapes thereby allowing the seal 200 to effectively seal the housing 101 within the specimen collection container or reservoir 302. The seal 200 may then be removed from the reservoir 302 or specimen collection container to retrieve the housing 101. Alternatively, the seal 200 is not removable from the reservoir 302 or specimen collection container after engaging the specimen collection container or reservoir 302. In other embodiments a tamper proof structure is either integrated into the seal 200 or is placed about the seal 200 after insertion into the sample collection container or reservoir 302 thereby preventing unwanted tampering with the housing 101 or assay strip. In further preferred embodiments, the housing 101 is permanently sealed inside the reservoir 302 or collection container by the seal 200 prior to the consumer receiving the present invention 100 thereby reducing consumer assembly.

Attachment Means

As shown and described herein, an attachment means 104 attaches the seal 200 to the top 102 of the housing 101 by one of several attachment means 104 such as but not limited to gluing, taping, micro-welding, welding, pinning, stapling, snapping, strapping, clipping and the like. The attachment means 104 functions to suspend or keep in place a housing 101 within the reservoir 302 or specimen collection container during the use of a specimen collection and assay container. In preferred embodiments of the present invention, the seal 200 is attached to the top 102 of the housing 101 by at least one prong on the top 102 of the housing 101. In alternative embodiments the attachment means is a structure or set of structures along the inner portion of the seal 200 able to clip a housing 101. A clip-like structure integrated into the seal 200 may be desirable when the housing 101 does not have a sufficiently large top 102 to include the at least one prong. In these alternative embodiments the housing 101 is pressed between the clip-like structure until the clip-like structure is able to suspend the housing 101. The housing may then be removed from the seal 200 by pulling the housing 101 from the seal 200 with sufficient force.

Specimen Collection Container

The present invention can be used with a variety of specimen collection containers 300 known in the art. It is preferable to use a specimen collection container 300 or device that is able to engage the seal 200 and optionally able to transfer a portion of a sample/specimen to a reservoir 302. Transfer may be by direct transfer such as by an aperture or may be by indirect transfer such as by a valve-like structure. In certain aspects of the present invention, the reservoir 302 may be a holding tank for a portion of the specimen or sample when the volume of the specimen or sample is too large or when assaying devices or assaying strips are to be used. Alternatively, the sample may be aliquoted into several specimen containers 300 when more than one assay is to be performed. FIG. 1 shows one example of a specimen collection container 300 able to be used with the present invention. Optionally, the specimen collection container 300 includes at least one reservoir 302 configured to accept a portion of the specimen and to reversibly engage the present invention 100, so as to allow the assaying of such portions of the specimen for an analyte.

Specimen

The present invention may detect an analyte from a variety of specimens. In a preferred embodiment the specimen to be collected is a biological specimen. Such biological specimens include but are not limited to a sample from a subject such as an animal or a human. A sample from a subject can be of any appropriate type, such as a sample of fluid, tissue, organ or a combination thereof. The biological specimen can also be a sample of other biological material, such as plants, bacteria, cell or tissue cultures, viruses and prions, or food, including food such as material derived from plants or animals or combinations thereof. The sample can be processed prior to introduction into the specimen collection container 300 or the specimen collection container 300 can include reagents for use in such processing. In the alternative, a sample and reagent can be combined within the specimen collection container 300. Such reagents can be used to process a sample, such as digesting solid samples with appropriate reagents such as chemicals, such as acids or bases, or with enzymes such as proteases. Other reagents can be used to extract analytes from a sample, such as extraction of antigens from biological entities, such as antigens from etiological agents such as bacteria, parasites, viruses or prions such as known in the art.

While a number of different biological specimens are suitable for collection by the specimen collection container, commonly collected specimens are biological samples, including but not limited to fluid sample including urine, blood, serum, saliva, semen, secretions including vaginal secretions, central nervous system fluids, lavages and the like. The specimen can also be an environmental sample, such as a sample of soil, water, wastewater, landfill or landfill leachate.

### EXAMPLE

A drug of abuse assay is performed by obtaining a specimen collection container having a reservoir. The housing having an encased assay strip for at least one particular drug of abuse is attached to a seal. The housing is placed within the reservoir and the seal engages the reservoir creating an air tight seal. A person in need of drug screening is given the collection container, referred to as a urine cup. The person goes to the restroom and urinates into the cup, closes the urine cup, and returns the urine cup to a technician. A portion of the urine is transferred into the reservoir by a means appropriate for the urine cup used. The seal prevents overloading of the assay strips by the forces exerted from the compressed air within the reservoir. Upon transfer to the reservoir, the urine comes into contact with the bottom of the housing within the sealed reservoir. The sample application zone of the assay strips of the assay device contact the urine through notches in the bottom of the housing of the assay device. Upon contact, a portion of the urine moves along the flow path of the assay strips of the test device including the reagent zone and detection zone by capillary action. A labeled mobilzable reagent binds the analyte during migration through the reagent zone and an immobilized reagent captures the analyte within the detection zone. The technician waits 5-10 minutes and then reads the assay results from the detection zone of the assay strips through the indicator windows of the housing. The technician reports the results of the drugs of abuse assay and disposes of the used urine cup in the appropriate biohazard waste.

We claim:

1) An analysis device able to reversibly engage a sample collection device, said analysis device comprising:
a) a housing, having a top end, a bottom end, an exterior and an interior;
b) an assay strip able to detect an analyte of interest from a specimen, said assay strip being positioned within said interior of said housing;
c) a seal able to seal said housing within said sample collection device; and
d) an attachment means able to attach said seal to said housing.

2) The analysis device of claim 1, wherein said analysis device is constructed at least in part from a material selected from the group consisting of metal, silicon, glass, ceramic, plastic and a polymer.

3) The analysis device of claim 1, wherein said bottom of said housing further comprises at least one notch, said at least one notch communicatively connecting said exterior and said interior of said housing.

4) The analysis device of claim 3, wherein said specimen enters said housing through said at least one notch.

5) The analysis device of claim 1, wherein said bottom of said housing further comprises a wick in fluid communication with said assay strip.

6) The analysis device of claim 5, wherein said wick comprises a bibulous material.

7) The analysis device of claim 1, wherein said assay strip is capable of performing at least one specific binding reaction.

8) The analysis device of claim 7, wherein said specific binding reaction comprises an immunoassay.

9) The analysis device of claim 1, wherein said assay strip is capable of performing an enzymatic reaction.

10) The analysis device of claim 1, wherein said assay strip is capable of performing a chemical reaction.

11) The analysis device of claim 1, wherein said analyte of interest is selected from the group consisting of a drug, a drug of abuse, a hormone, a protein, a nucleic acid molecule, an etiological agent and a specific binding member.

12) The analysis device of claim 1, wherein said specimen is a liquid specimen.

13) The analysis device of claim 1, wherein said specimen is a biological specimen.

14) The analysis device of claim 1, wherein said specimen is urine, blood, serum or spinal fluid.

15) The analysis device of claim 1, wherein said seal comprises at least one rib.

16) The analysis device of claim 1, wherein said at least one rib is deformable.

17) The analysis device of claim 1, wherein said seal is constructed at least in part from a material selected from the group consisting of metal, silicon, glass, ceramic, plastic and a polymer.

18) The analysis device of claim 1, wherein said seal snugly seals said sample collection device.

19) The analysis device of claim 1, wherein said seal hermetically seals a reservoir portion of a sample collection device.

20) The analysis device of claim 19, wherein said analysis device is reversibly engaged within said reservoir.

21) The analysis device of claim 1, wherein said attachment means comprises at least one prong.

22) The analysis device of claim 1, wherein said attachment means comprises gluing, taping, micro-welding, welding, pinning, stapling, snapping or strapping.

23) The analysis device of claim 1, wherein said housing comprises an orientation means able to vertically orientate said analysis device when said analysis device is engaged to said sample collection device.

24) The analysis device of claim 23, wherein said orientation means comprises a pin.

25) The analysis device of claim 23, said orientation means is constructed from a material selected from the group consisting of metal, silicon, glass, ceramic, plastic and a polymer.

26) The analysis device of claim 23, wherein said orientation means is integral with said housing.
